(19) United States
(12) Patent Application Publication
(10) Pub. No.: US 2018/0185190 A1
(43) Pub. Date: Jul. 5, 2018

(54) THERAPEUTIC SHOULDER WRAP

(71) Applicant: Irvin Tilley, Orem, UT (US)

(72) Inventor: Irvin Tilley, Orem, UT (US)

(21) Appl. No.: 15/399,007

(22) Filed: Jan. 5, 2017

(51) Int. Cl. A61F 7/02

(52) U.S. Cl. CPC A61F 7/02; A61F 2007/108; A61F 2007/0231

(57) ABSTRACT

A shoulder wrap for therapeutic use that includes: a shoulder cover; a first wrap extending from the shoulder cover; and then a second wrap extending from the shoulder cover. A first receptacle and a second receptacle reside within the shoulder cover adapted to receive a first cooling element and a second cooling element, respectively. Preferably, a sleeve extends from the shoulder cover adapted to receive a user's arm. In one particular embodiment, the cooling element may be a frozen water bottle. Other features of the shoulder wrap include a first strap and a second strap adapted to further secure the shoulder wrap around a user's shoulder area, wherein hook and loop fasteners are used to secure the shoulder straps into a secure position.

### BACKGROUND OF THE INVENTION

### Field of Invention

The present invention relates to a therapeutic shoulder wrap that is used to aid in the healing of a user's shoulder.

### Description of Related Art

Many individuals suffer orthopedic injuries on a yearly basis. Orthopedic injuries include injuries to the knees, hips, ankles, elbows and other joints of the body. Many times the injuries involve swelling and pain. As a result patients must go through various types of strategies to reduce swelling and inflammation related to those injuries. This strategy normally includes both the administration of medicine and application of topical treatment over the affected area. One common method to reduce inflammation and swelling involves the application of a cold compress. The cold compress may come in various forms in the form of an ice bag, frozen pads and other means to keep a cooling effect over the affected area. Some individuals freeze bottles of water or actually use frozen food material such as frozen bags of vegetables over a particular area. One area of application that is difficult to apply a steady cooling compress is the shoulder area. Due to the shape of the shoulder and the general inconvenience it can be difficult to apply an ice pack over the shoulder for an extended period of time. Therefore it would be advantageous to have a wrap, which is designed to particularly apply a cold compress to the shoulder area of a user.

### SUMMARY OF THE INVENTION

The present invention relates to a shoulder wrap for therapeutic use that includes: a shoulder cover; a first wrap extending from the shoulder cover; and then a second wrap extending from the shoulder cover. A first receptacle and a second receptacle reside within the shoulder cover adapted to receive a first cooling element and a second cooling element, respectively. Preferably, a sleeve extends from the shoulder cover adapted to receive a user's arm. In one particular embodiment, the cooling element may be a frozen water bottle. Other features of the shoulder wrap include a first strap and a second strap adapted to further secure the shoulder wrap around a user's shoulder area, wherein hook and loop fasteners are used to secure the shoulder straps into a secure position.

### BRIEF DESCRIPTION OF DRAWINGS

FIG. 1 depicts an inside view of a shoulder wrap in accordance with the present invention.

FIG. 2 depicts the inside view showing the insertion of cooling elements into receptacles of the shoulder wrap.

FIG. 3 shows an outside view of the shoulder wrap in accordance with the present invention.

FIG. 4A depicts s a first side view of the shoulder wrap showing a sleeve portion.

FIG. 4B shows a second side view of the shoulder wrap with the sleeve portion.

FIG. 5 shows the first side view of the shoulder wrap with the application of straps.

FIG. 6 shows a second side view of the shoulder wrap with the inclusion of straps.

### DETAILED DESCRIPTION

The present invention relates to a shoulder wrap that wraps securely around an individual's shoulder to apply a cold compress treatment onto the shoulder area. The shoulder wrap according to the present invention includes a main portion that covers the shoulder that includes receptacles to receive cooling elements. The cooling elements are placed within the receptacles and then the shoulder wrap is placed over the shoulder of the user. Wraps extending from each side of the cover wrap around the torso of the user to secure the shoulder cover into place. Another feature of the shoulder wrap in accordance with the present invention is a sleeve that extends from the cover that allows the user to place their arm there through placing the shoulder wrap in a secure position over the user's shoulder. In addition to the wraps that extend around the torso include straps that may be implemented to further secure the shoulder wrap into place over a user's shoulder.

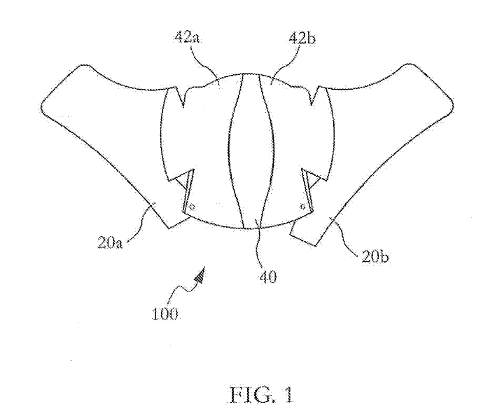

A shoulder wrap 100 according to the present invention is depicted in FIG. 1. The shoulder wrap 100 includes a shoulder cover 40 that includes receptacles 42a, 42b. The view shown in FIG. 1 is an inside view of the shoulder cover 40 which includes a first wrap 20a and a second wrap 20b extending from the shoulder cover 40. The receptacles 42a, 42b receive cooling elements that are placed within the receptacles 42a, 42b and sealed therein to apply the shoulder cover 40 over a user's shoulder area.

In one particular embodiment the cooling elements may include frozen water bottles therefore providing a convenient cooling element to insert within the receptacles. In reference to

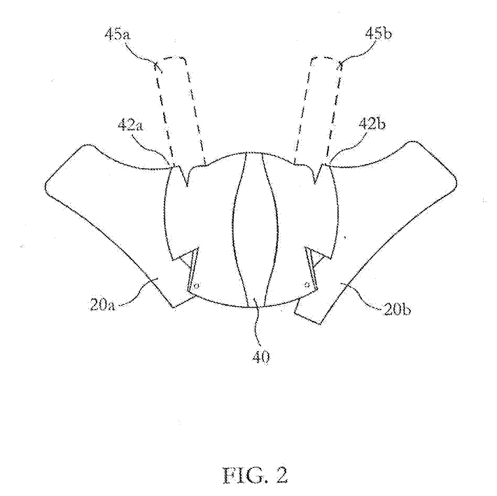

FIG. 2, cooling elements 45a, 45b are shown extending from the receptacles 42a, 42b respectively. Cooling elements 45a, 45b are preferably removable and insertable into receptacles 42a, 42b for use by the user.

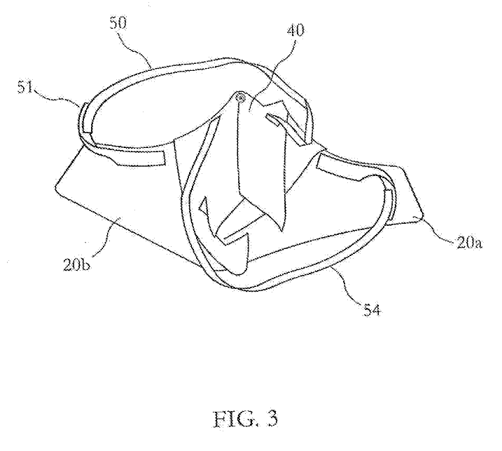

An outside view of the shoulder wrap 100 is shown in FIG. 3 where shoulder cover 40 is shown with the wraps 20a, 20b extending therefrom. Further shown in FIG. 3 are straps 50, 54. The strap 50 attaches to a adjoining strap 51 to help secure the shoulder wrap 100 onto a user's shoulder. These straps attach and are secured, in one particular embodiment, through the use of hook and loop connectors to secure the shoulder wrap around the user's shoulder.

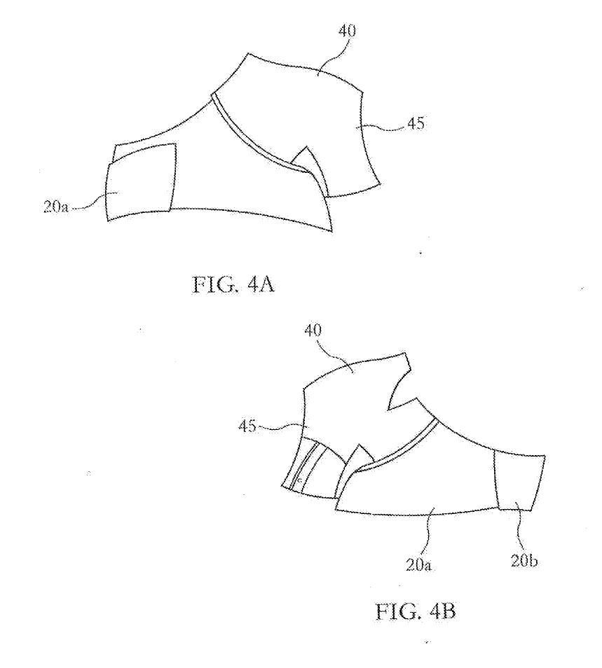

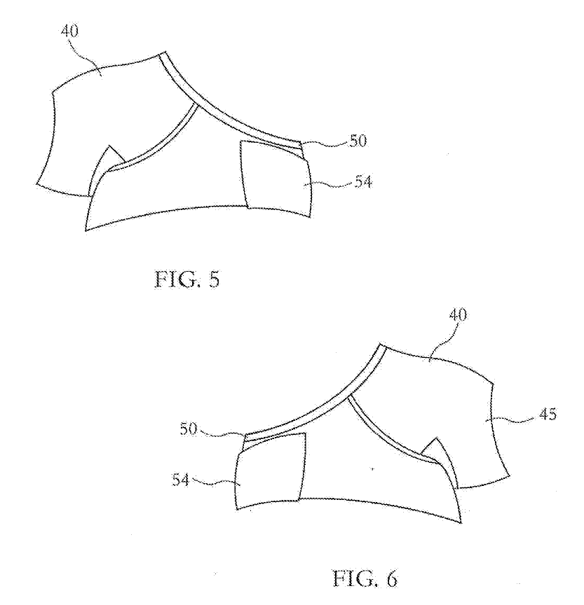

FIGS. 4A and 4B show two side views of the shoulder cover 40 showing the sleeve 45 extending from shoulder cover 40. The sleeve 45 acts to secure the cover 40 in place through the insertion of a user's arm. Further views of the shoulder cover 40 are shown in FIGS. 5 and 6 where straps 50 and 54 are secured in a closed and attached position to the opposing respective shoulder wrap 20a, 20b. In this closed position these straps secure the shoulder wrap 100 around the user's shoulder.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A shoulder wrap for therapeutic use comprising:
a shoulder cover;
a first wrap extending from the shoulder cover;
a second wrap extending from the shoulder cover;
a first receptacle within the shoulder cover adapted to receive a first cooling element;
a second receptacle within the shoulder cover adapted to receive a second cooling element; and
a sleeve extending from the shoulder cover adapted to receive a user's arm.

2. The shoulder wrap according to claim 1, wherein the cooling element is a frozen water bottle.

3. The shoulder wrap according to claim 1 further including a first strap and a second strap adapted to further secure the shoulder wrap around a user's shoulder area.

4. The shoulder wrap according to claim 1, wherein hook and loop fasteners are used to secure the shoulder straps into a secure position.
